(19) United States
(12) Patent Application Publication
(10) Pub. No.: US 2014/0296651 A1
(43) Pub. Date: Oct. 2, 2014

(54) System and Method for Monitoring Physiological Characteristics

(71) Applicant: Robert T. Stone, Sunnyvale, CA (US)

(72) Inventor: Robert T. Stone, Sunnyvale, CA (US)

(21) Appl. No.: 13/854,280

(22) Filed: Apr. 1, 2013

(51) Int. Cl. A61B 5/00; A61B 5/01; A61B 5/08

(52) U.S. Cl. CPC A61B 5/6804; A61B 5/08; A61B 5/01
USPC 600/301; 600/388

(57) ABSTRACT

A wearable physiological monitoring system comprising a garment that is configured to cover at least the chest region and the upper back of a wearer, a stretchable circumferential band that is attachable to the garment, the stretchable band including a respiration detection system that is configured to detect axial chest wall displacements of the wearer and integral signal transmission conductors, an electronics module that is releasably attachable to said garment and programmed to control the respiration detection system, process signals therefrom, and wirelessly transmit the processed signals, and a self-aligning magnetic connection system that is configured to removeably couple the electronics module to said band and, thereby, the signal transmission conductors.

### FIELD OF THE INVENTION

The present invention relates to systems and methods for monitoring physiological characteristics of a subject. More particularly, the present invention relates to apparatus, systems and methods for determining a plurality of physiological characteristics; particularly, respiratory characteristics, in real time.

### BACKGROUND OF THE INVENTION

In medical diagnosis and treatment of a subject, it is often necessary to assess one or more physiological characteristics; particularly, respiratory characteristics. A key respiratory characteristic is respiratory air volume (or tidal volume).

Various conventional methods and systems have thus been employed to measure (or determine) tidal volume. One method includes having the patient or subject breathe into a mouthpiece connected to a flow rate measuring device. Flow rate is then integrated to provide air volume change.

As is well known in the art, there are several drawbacks and disadvantages associated with employing a mouthpiece. A significant drawback associated with a mouthpiece and nose-clip measuring device is that the noted items cause changes in the monitored subject's respiratory pattern (i.e., rate and volume). Tidal volume determinations based on a mouthpiece and nose-clip are, thus, often inaccurate.

Other conventional devices for determining tidal volume include respiration monitors. Illustrative are the systems disclosed in U.S. Pat. Nos. 3,831,586 and 4,033,332.

Although the noted systems eliminate many of the disadvantages associated with a mouthpiece, the systems do not, in general, provide an accurate measurement of tidal volume. Further, the systems are typically only used to signal an attendant when a subject's breathing activity changes sharply or stops.

A further means for determining tidal volume is to measure the change in size (or displacement) of the rib cage and abdomen, as it is well known that lung volume is a function of these two parameters. A number of systems and devices have been employed to measure the change in size (i.e., circumference) of the rib cage (and/or abdomen), including pneumobelts and respiratory inductive plethysmograph (RIP) belts.

RIP belts are a common means employed to measure changes in the cross-sectional areas of the rib cage and abdomen. RIP belts include conductive loops of wire that are coiled and sewed into an elastic belt. As the coil stretches and contracts in response to changes in a subject's chest cavity size, a magnetic field generated by the wire changes. The output voltage of an RIP belt is generally related to changes in the expanded length of the belt and, thus, changes in the enclosed cross-sectional area.

In practice, measuring changes in the cross-sectional areas of the abdomen can increase the accuracy of RIP belt systems. To measure changes in the cross-sectional areas of the rib cage and abdomen, one belt is typically secured around the mid-thorax and a second belt is typically placed around the mid-abdomen.

RIP belts can also be embedded in a garment, such as a shirt or vest, and appropriately positioned therein to measure rib cage and abdominal displacements, and other anatomical and physiological parameters. Illustrative is the system disclosed in U.S. Pat. No. 6,551,252.

There are, however, several drawbacks associated with most RIP belt systems. A major drawback is that RIP belts are typically expensive in terms of material construction and in terms of the electrical and computing power required to operate them.

In an attempt to rectify the drawbacks associated with RIP belt systems, various magnetometer-based systems have been recently developed to measure displacements of the rib cage and abdomen and, thereby, various respiratory parameters. The noted magnetometer-based systems typically comprise at least one pair of tuned air-core magnetometers or electromagnetic coils. The paired magnetometers are responsive to changes in a spaced distance therebetween; the changes being reflected in the difference between the strength of the magnetic field between the paired magnetometers.

To measure changes in (or displacement of) the anteroposterior diameter of the rib cage, a first magnetometer is typically placed over the sternum at the level proximate the 4th intercostal space and the second magnetometer is placed over the spine at the same level.

In some magnetometer-based systems, additional magnetometers are employed to increase the accuracy of the system. For example, to measure changes in the anteroposterior diameter of the abdomen, a third magnetometer can be placed on the abdomen at the level of the umbilicus and a fourth magnetometer can be placed over the spine at the same level. Illustrative is the magnetometer-based system disclosed in U.S. Pub. No. 2011/0054271.

Over the operational range of distances, the output voltage is linearly related to the distance between two magnetometers; provided, the axes of the magnetometers remain substantially parallel to each other. As rotation of the axes can change the voltage, the magnetometers are typically secured to the subject's skin in a parallel fashion, whereby rotation due to the motion of underlying soft tissue is minimized.

To overcome the problems associated with direct attachment of magnetometers to the skin of a subject, some magnetometer-based systems are configured to embed or carry the magnetometers (and associated physiological sensors) in a wearable garment, such as a shirt or vest. The wearable monitoring garment also facilitates repeated and convenient positioning of magnetometers at virtually any appropriate (or desired) position on a subject's torso.

A major drawback and disadvantage associated with many garment based magnetometer systems is that the wires that are employed to effectuate communication by and between the magnetometers and other electronic components, e.g., sensors, are typically disposed outside of the garment or disposed partially or wholly within the garment seams. As a result, the wires can, and often will, catch and tangle on objects. The wires also reduce mobility and add weight. Further, the wires are not, in general, washable or resistant to corrosion. Such a design is, thus, not very robust.

In an effort to overcome the drawbacks associated with exposed wires, various systems have been developed that employ conductive garment fabrics, wherein electronic circuits and/or data and power conductors are integrated within the garment itself. Illustrative are the garment based systems disclosed in U.S. Pat. Nos. 6,080,690 and 5,906,004.

There are, however, several drawbacks associated with such systems. For example, routing of the data or power between electronic components is limited without extensive formation of electrical junctions in the fabric—a very cumbersome manufacturing process. In addition, such garments are also uncomfortable and cannot withstand repeated wash cycles.

A further drawback and disadvantage of systems employing conductive garment fabrics, as well as exposed wiring, is that it is difficult to achieve an effective or secure mechanical and electrical interconnection between external or portable modules or subsystems, e.g., processing or control unit, and the integrated circuitry and/or electronic components.

It would thus be desirable to provide an improved garment based physiological monitoring system and method that (i) accurately measures one or more physiological characteristics associated with a user or wearer; particularly, respiratory characteristics, (ii) does not require the user to secure electrodes to their body or to use any conductive gels, (iii) does not include any exposed electrical circuitry, (iv) does not include any wires which must be connected or routed by the wearer, (v) does not interfere with the activities of or duties carried out by the user, and (vi) is aesthetically pleasing.

It is therefore an object of the present invention to provide an improved garment based physiological monitoring system and method that accurately (i) monitors and detects changes in (or displacements of) the anteroposterior diameters of the rib cage, and axial displacements of the chest wall, and (ii) determines anatomical and physiological information associated with the monitored subject as a function of the signals reflecting the noted anatomical displacements.

It is another object of the present invention to provide an improved garment based physiological monitoring system and method that accurately measures multiple physiological characteristics associated with a user or wearer.

It is another object of the present invention to provide an improved garment based physiological monitoring system and method that does not require the user to secure electrodes to his/her body or to use any conductive gels.

It is another object of the present invention to provide an improved garment based physiological monitoring system and method that does not include any exposed electrical circuitry.

It is another object of the present invention to provide an improved garment based physiological monitoring system and method that does not include any wires which must be connected or routed by the wearer.

It is another object of the present invention to provide an improved garment based physiological monitoring system and method that includes reliable and effective means to connect external modules, e.g. processing units.

It is another object of the present invention to provide an improved garment based physiological monitoring system and method that does not interfere with the activities of or duties carried out by the user.

It is another object of the present invention to provide an improved garment based physiological monitoring system and method that requires minimal or no preparation prior to or after donning the garment.

It is another object of the present invention to provide an improved garment based physiological monitoring system and method that is easy to use.

It is another object of the present invention to provide an improved garment based physiological monitoring system that is aesthetically pleasing.

### SUMMARY OF THE INVENTION

The present invention is directed to an improved physiological monitoring system and associated method. In a preferred embodiment of the invention, the system includes a garment that is configured to cover at least the chest region and upper back of a wearer (or user). The garment includes a stretchable circumferential band having a respiration detection system and integral signal transmission conductors associated therewith.

In some embodiments, the system further includes one or more additional physiological sensors that are in communication with the signal transmission conductors.

In a preferred embodiment of the invention, the respiration detection system includes two magnetic coils or magnetometers that are configured and positioned (via the garment band) to monitor and detect changes in (or displacements of) the anteroposterior diameters of a user's rib cage, and axial displacements of the chest wall of the user.

In a preferred embodiment of the invention, the system further includes an electronics module that is configured to be releasably attached to the garment band.

In a preferred embodiment of the invention, the electronics module includes at least a processing system and data transmission system.

In a preferred embodiment, the module processing system includes programs, instructions and associated algorithms and parameters to control the respiration detection system and the function thereof, and the transmission and receipt of signals therefrom.

The module processing system is also preferably programmed and adapted to retrieve and process transmissions or signals from the respiration detection system, and to determine anatomical and physiological information associated with a monitored subject (as a function of the signals), including at least one respiratory characteristic.

In a preferred embodiment, the data transmission system includes a transmitter that is preferably configured to wirelessly transmit the processed signals.

In a preferred embodiment of the invention, the monitoring system includes a unique self-aligning magnetic connection system that facilitates communication by and between the band and, hence, signal transmission conductors, respiration detection system, and additional physiological sensors (if employed), and the electronics module.

In a preferred embodiment, the band includes a first magnetic connector subsystem, which is accessible from outside the garment, and the electronics module includes a second magnetic connector subsystem that mates with the first magnetic connector subsystem.

In some embodiments, the monitoring system further includes a remote display unit having a receiver that is programmed and configured to receive the transmitted processed signals. The remote display is also programmed to display the received processed signals on the display unit.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of a physiology monitoring system, in accordance with the invention;

FIG. 2 is a schematic illustration of a paired electromagnetic coil, i.e. magnetometer, arrangement, in accordance with the invention;

FIG. 3 is a side view of a subject, showing the position of the paired electromagnetic coil arrangement shown in FIG. 2 on the subject, in accordance with one embodiment of the invention;

FIG. 4 is a perspective view of the subject, showing the position of a first electromagnetic coil on the front of the subject, in accordance with one embodiment of the invention;

FIG. 5 is a plane view of the subject's back, showing the position of a second electromagnetic coil thereon, in accordance with one embodiment of the invention;

FIG. 6 is a perspective view of one embodiment of a wearable physiological monitoring system fitted on a subject, in accordance with the invention;

FIG. 7 is a top plane view of one embodiment of a stretchable system band that is configured for attachment to the wearable physiological monitoring system shown in FIG. 6, in accordance with the invention;

FIG. 8 is a perspective view of one embodiment of an electronics module, in accordance with the invention;

FIG. 9 is a rear plane view of the electronics module shown in FIG. 8, showing a module magnetic connector subsystem, in accordance with one embodiment of the invention;

FIG. 10 is another perspective view of the wearable physiological monitoring system shown in FIG. 6, showing a band magnetic connector subsystem that is configured to mate with the module magnetic connector subsystem shown in FIG. 9, in accordance with one embodiment of the invention;

FIG. 11 is a further perspective view of the wearable physiological monitoring system shown in FIG. 6, showing the electronics module shown in FIGS. 8 and 9 attached thereto, in accordance with one embodiment of the invention;

FIG. 12 is an assembled perspective view of one embodiment of a magnetic connector, in accordance with the invention;

FIG. 13 is an exploded perspective view of the magnetic connector shown in FIG. 12, in accordance with one embodiment of the invention;

FIG. 14 is a top plane view of one embodiment of a magnetic connector top member, showing conductive pads on the engagement end thereof, in accordance with one embodiment of the invention;

FIG. 15 is a top plane view of one embodiment of a magnetic connector bottom member, showing mating conductive pads on the base thereof, in accordance with one embodiment of the invention; and

FIGS. 16 and 17 are side plane, partial sectional views of the magnetic connector shown in FIG. 13, showing the engagement and disengagement directions, in accordance with one embodiment of the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a sensor signal” includes two or more such signals and the like.

### DEFINITIONS

The terms “respiratory parameter” and “respiratory characteristic”, as used herein, mean and include a characteristic associated with the respiratory system and functioning thereof, including, without limitation, breathing frequency, tidal volume, inspiration volume, expiration volume, minute ventilation, inspiratory breathing time, expiratory breathing time, and flow rates (e.g., rates of change in the chest wall volume).

The terms “respiratory parameter” and “respiratory characteristic” further mean and include parameters associated with ventilation mechanics from synchronous or asynchronous movements of the chest wall compartments.

According to the present invention, flow rates and respiratory accelerations can be determined from a volume signal. Further, numerous inferences regarding ventilation mechanics can be drawn from the degree of asynchrony in movement occurring amongst the discrete compartments that make up the chest wall.

The terms “respiratory system disorder”, “respiratory disorder”, and “adverse respiratory event”, as used herein, mean and include any dysfunction of the respiratory system that impedes the normal respiration or ventilation process.

The terms “physiological parameter” and “physiological characteristic”, as used herein, mean and include, without limitation, electrical activity of the heart, electrical activity of other muscles, electrical activity of the brain, pulse rate, blood pressure, blood oxygen saturation level, skin temperature, and core temperature.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

It is understood that although the physiological monitoring systems and associated methods of the invention are described herein in connection with monitoring physiological parameters and characteristics in a human body, the invention is in no way limited to such use. The physiological monitoring systems and associated methods of the invention can also be employed to monitor physiological parameters in non-human bodies.

It is also understood the although the present invention is described herein in terms of magnetometers and magnetometer systems, other types of sensor systems capable of measuring changes in distance between two or more sensors in the system can be used in place of, or in addition to, magnetometers. The invention is thus not limited to the use of electromagnetic coils or magnetometers to acquire signals representing measured changes in the anteroposterior diameters of the rib cage and/or axial displacement of the chest wall. Indeed, various additional means and devices that can be readily adapted to measure the noted anatomical parameters can be employed within the scope of the invention. Such means and devices include, without limitation, Hall effect sensors.

Wireless sensors with the capability of measuring time delay in a signal sent from one sensor to another and, thereby, determine the distance between the two sensors can also be substituted for or provided in addition to magnetometers in accordance with the present invention.

The physiological monitoring systems and associated methods of the invention can also be employed in non-medical contexts, such as determining volumes and/or volume changes in extensible bladders used for containing liquids and/or gasses.

As indicated above, the present invention is directed to an improved physiological monitoring system and associated method. In a preferred embodiment, the monitoring system includes a garment that is configured to cover at least the chest region and upper back of a wearer, and includes a stretchable circumferential band.

In a preferred embodiment, the band is attached to the interior portion of the garment. According to the invention, the band can be permanently attached to the garment or removeably secured to the garment, e.g. via a zipper or Velcro® system.

Referring now to FIG. 1, in a preferred embodiment of the invention, the monitoring system (denoted “100”) includes a respiration detection system 20, signal transmission conductors 10 and an electronics module 40. As illustrated in FIG. 1, the system 100 further includes a power source 60.

As discussed in detail herein, in a preferred embodiment of the invention, the respiration detection system 20 includes a pair of electromagnetic coils or magnetometers that are secured and positioned by the stretchable circumferential garment band.

In a preferred embodiment, the system 100 further includes an electronics module 40. The module 40 preferably includes a processing system, which is programmed and configured to control the respiration detection system 20 and the function thereof, and the transmission and receipt of signals therefrom.

The module processing system is also preferably programmed and adapted to retrieve and process transmissions or signals from the respiration detection system 20, and to determine anatomical and physiological information associated with a monitored subject (as a function of the signals), including at least one respiratory characteristic.

In a preferred embodiment, the electronics module 40 further includes a data transmission system having a transmitter that is programmed and configured to wirelessly transmit processed signals to a remote signal receiving device, e.g., a base module or a hand-held electronic device, such as a smart phone, tablet, computer, etc.

In some embodiments of the invention, the monitoring system 100 further includes one or more physiological sensors, such as a pulse oximeter (Sₚ0₂) 45a or core body temperature sensor 45b, which are in communication with the signal transmission conductors 10.

In a preferred embodiment, the monitoring system 100 further includes a self-aligning magnetic connection system 30, which facilitates connection and thereby, signal communication by and between the garment band and, hence, signal transmission conductors 10, respiration detection system 20 and additional physiological sensors (if employed), and the electronics module 40.

In some embodiments, the monitoring system 100 further includes a remote display unit 102 having a receiver that is programmed and configured to receive the transmitted processed signals. The remote display unit 102 also includes a second processing system that is programmed and to display the received processed signals on the display unit 102.

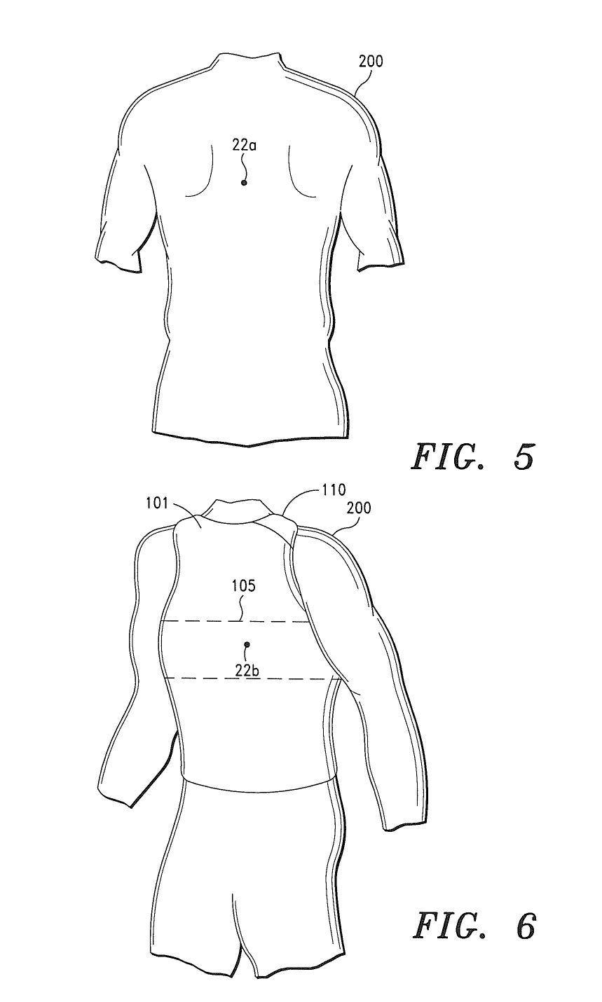

Referring now to FIGS. 1-11, an exemplary embodiment of a physiological monitoring system of the invention will be described in detail. As indicated above, the monitoring system 100 is adapted to (i) monitor and detect changes in (or displacements of) the anteroposterior diameters of the rib cage, and axial displacement of the chest wall, and (ii) determine anatomical and physiological information associated with the monitored subject as a function of the signals reflecting the noted anatomical displacements.

In some embodiments, the monitoring system 100 is further adapted to monitor one or more additional physiological characteristics associated with the monitored subject.

Referring first to FIGS. 1, 6 and 7, the physiological monitoring system 100 preferably includes a garment (denoted generally “101) that includes a stretchable circumferential band 105. In a preferred embodiment, the band 105 is attached to the interior portion of the garment 101, as shown in FIG. 6. As indicated above, the band 105 can be permanently attached to the garment or removeably secured to the garment 101, e.g. via a zipper or Velcro® system.

According to the invention, the garment 105 can comprise various conventional fabrics having fibers of variable loft and thickness. In some embodiments of the invention, the garment comprises a form fitting garment constructed of Lycra® or like material.

In some embodiments of the invention, at least one of the shoulder portions 110 of the garment 101 comprises a two-piece portion, i.e. an over-lapping strap configuration, to facilitate easy placement of the garment 101 on a wearer, e.g., elderly user. In the noted embodiments, the two-piece portion includes a conventional Velcro® system or hooks or snaps to secure the ends of the over-lapping strap after the garment 101 is positioned on the wearer's body.

In a preferred embodiment of the invention, the garment 101 includes at least one opening, which is preferably disposed in the front of the garment 101, for releasable attachment of electronic components, e.g. the electronics module 40 (discussed below), diagnostic devices, etc., to the garment band 105.

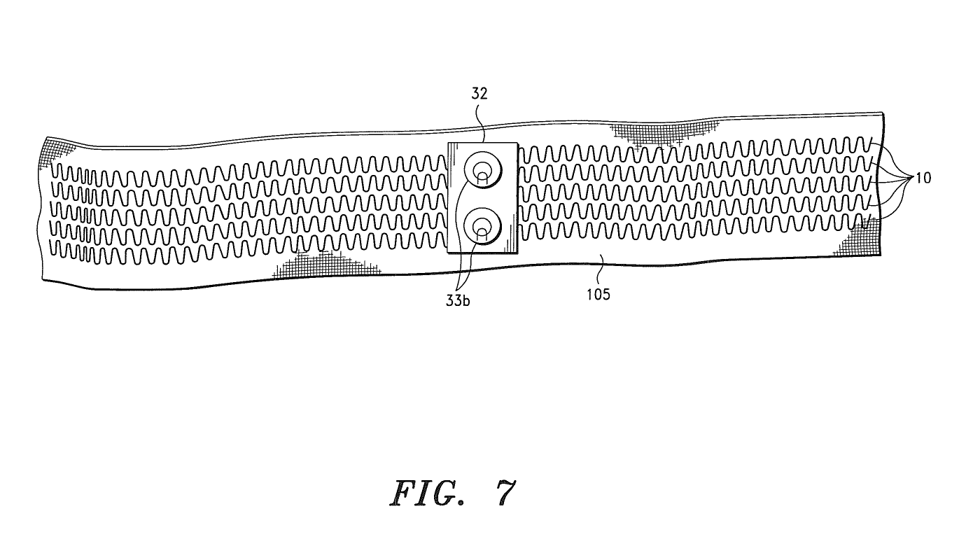

In a preferred embodiment, the garment band 105 includes the respiration detection system 20 and integral signal transmission conductors 10 in a flexible configuration thereon (see FIG. 7). The system 100 further includes a power source 60, such as a battery.

In some embodiments of the invention, the signal transmission conductors 10 comprise conductive fabric. In some embodiments, the signal transmission conductors 10 comprise a thin linear member, e.g. thread or chord, which is wrapped with a conductive wire. Preferably, the linear member comprises a stretchable member, i.e. is at least partially constructed of a stretchable material, and the wire is spirally wrapped around the stretchable member.

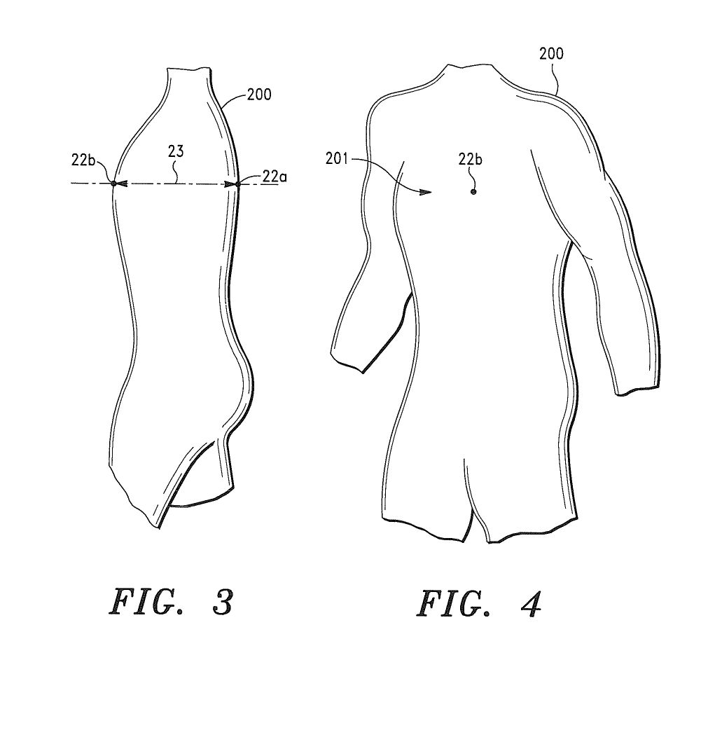

Retelling now to FIGS. 3-6, the respiration detection system 20 includes a pair of electromagnetic coils or magnetometers 22a, 22b that are secured and positioned by the stretchable band 105. In some embodiments of the invention, the band 105 includes pockets that are configured to removeably receive and, hence, position the magnetometers 22a, 22b. In some embodiments, the magnetometers 22a, 22b are permanently attached to the band 105.

In a preferred embodiment, the paired magnetometers 22a, 22b are configured and positioned to monitor and detect changes in (or displacements of) the anteroposterior diameters of the rib cage 201, and axial displacement of the chest wall of a subject 200, i.e. user of the system 100.

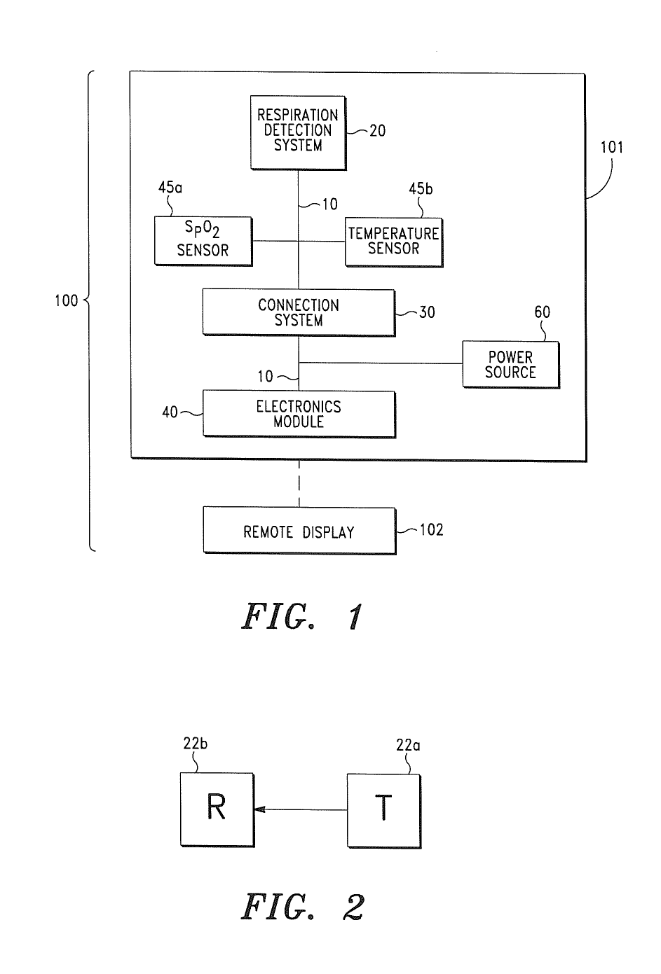

In the illustrated embodiment, the first magnetometer 22a comprises a transmitter magnetometer and the second magnetometer 22b comprises a receiving magnetometer 22b (see FIG. 2).

As indicated above and illustrated in FIG. 3-5, the magnetometers 22a, 22b are preferably disposed in-plane (denoted by line “23”). Preferably, a first magnetometer, i.e. 22a or 22b, is disposed on the front of the subject 200 proximate the subject's umbilicus and a second paired magnetometer is disposed on the back of the subject 200 proximate the same axial position.

In the illustrated embodiment, the first magnetometer 22a is disposed on the back of the subject 200 and the second magnetometer 22b is disposed on the front of the subject 200.

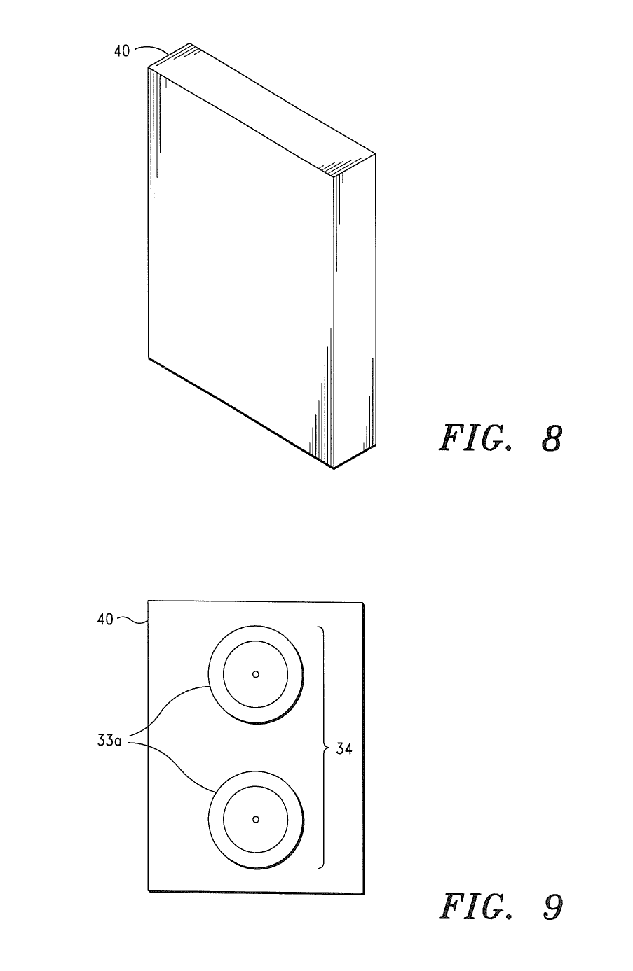

Referring now to FIGS. 8-11, the system 100 further includes an electronics module 40, which, as discussed below, is configured to be releasably attached to the band 105. In a preferred embodiment of the invention, the electronics module 40 includes at least a processing system and data transmission system.

Preferably, the module processing system includes programs, instructions and associated algorithms and parameters to control the respiration detection system 20 and, hence, the paired magnetometers 22a, 22b and the function thereof, and the transmission and receipt of signals therefrom, as well as the data transmission system.

The module processing system is also preferably programmed and adapted to retrieve and process transmissions or signals from the respiration detector subsystem 20, i.e. signals reflecting changes in the magnetometer fields (and, hence, changes in spaced distances between the paired magnetometers 22a, 22b), and to determine anatomical and physiological information associated with the monitored subject (as a function of the signals), including at least one respiratory characteristic, more preferably, a plurality of respiratory characteristics.

In some embodiments of the invention, the processing system (or the remote display unit 102, discussed below) also includes a “rules set” that includes a rule in which an alert signal is transmitted if the signals from the respiration detection system 20 indicate that a breathing rate or other physiological parameter that is being monitored is outside a predetermined range.

In a preferred embodiment, the data transmission system includes a transmitter that is programmed and configured to wirelessly transmit processed signals to a remote signal receiving device, e.g., a base module or a hand-held electronic device, such as a smart phone, tablet, computer, etc.

In some embodiments, the electronics module 40 further includes a GPS or other position detection subsystem, and/or a motion detector, such as an accelerometer.

In some embodiments, the module 40 also includes display means and is programmed and configured to display received and/or processed signals.

In some embodiments, the system 100 further includes one or more additional physiological sensors, such as an ECG, temperature or SpO₂ sensor. In at least one embodiment, the system includes a temperature sensor.

As indicated above, in a preferred embodiment, the monitoring system 100 further includes a self-aligning magnetic connection system 30, which facilitates connection and thereby, signal communication by and between the band 105 and, hence, signal transmission conductors 10, respiration detection system 20 and additional physiological sensors (if employed), and the electronics module 40.

In a preferred embodiment of the invention, the magnetic connection system 30 includes cooperating magnetic connector subsystems. Referring to FIG. 7, in a preferred embodiment, the garment band 105 includes a first magnetic connector subsystem 32. The first magnetic connector subsystem 32 preferably includes at least one, more preferably, a plurality of conductive pads (or pins, e.g. pogo pins), which are in communication with the signal transmission conductors 10.

Referring now to FIG. 9, the electronics module 40 includes a second magnetic connector subsystem 34 that is configured to mate with the first magnetic connector subsystem 32. The second magnetic connector subsystem 34 similarly includes at least one, more preferably, a plurality of conductive pads or pins that are configured and aligned to mate with the first magnetic connector pads (or pins) when the first and second magnetic connector subsystems 32, 34 are engaged.

The magnetic connector subsystems 32, 34 thus facilitate communication and, thereby, signal transmission by and between the electronics module 40 and band 105 and, hence, signal transmission conductors 10 (and electronics associated therewith) when the magnetic connector subsystems 32, 34 are engaged.

In some embodiments, the system 100 further includes a remote display unit 102. In a preferred embodiment, the remote display unit includes a receiver that is configured and programmed to receive the transmitted processed signals and a second processing system that is programmed to display received processed signals on the display unit 102.

In the noted embodiments, the electronics module 40 also includes a receiver for receiving communications from the remote display unit 102.

According to the invention, the system 100 can further include a portal, such as a website accessible over a network (that is responsive to the remote display unit 102), to display and store the processed signals.

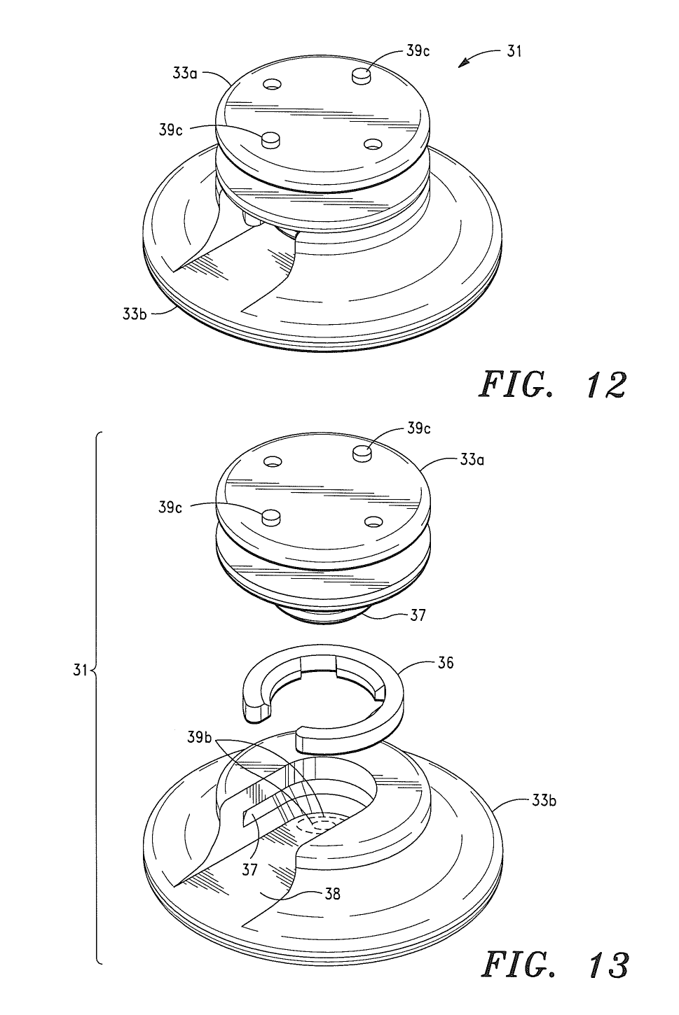

Referring now to FIGS. 12-17, the unique first and second magnetic connector subsystems 32, 34 that are associated with the connection system 30 and electronics module 40 of the invention will be described in detail. Referring first to FIGS. 12 and 13, there is shown a magnetic connector 31 that is an integral component of the first and second magnetic connector systems 32, 34.

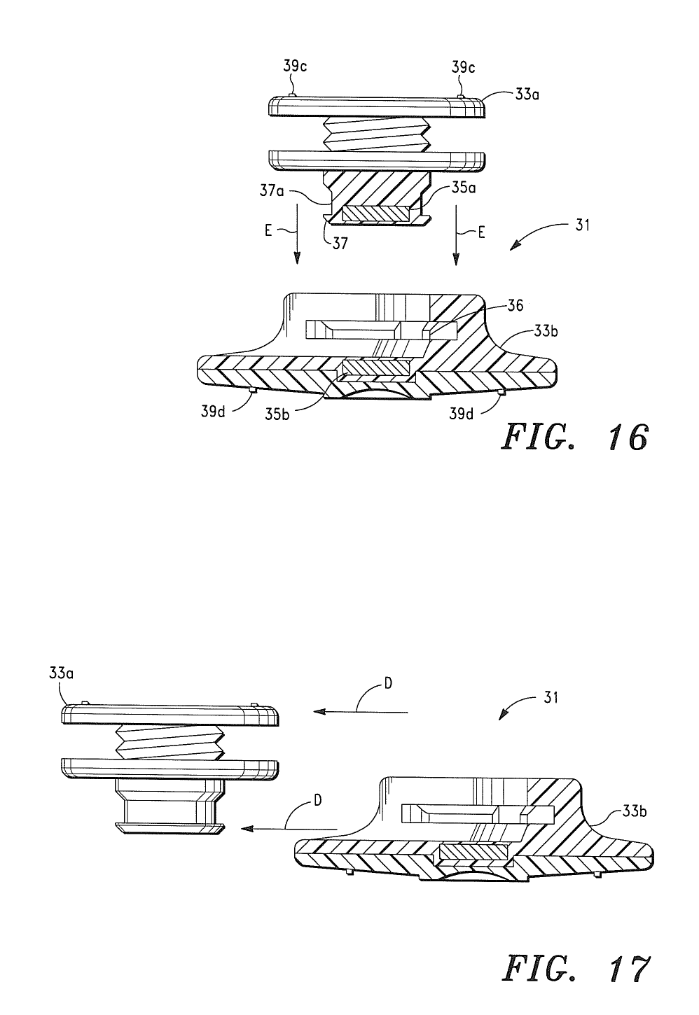

As illustrated in FIG. 13, the connector 31 includes a top (or male) member 33a and a bottom (or female) member 33b. Referring to FIG. 16, the top member 33a includes a first magnet 35a having a first polarity, and the bottom member 33b includes a second magnet 35b having a second (or opposite) polarity.

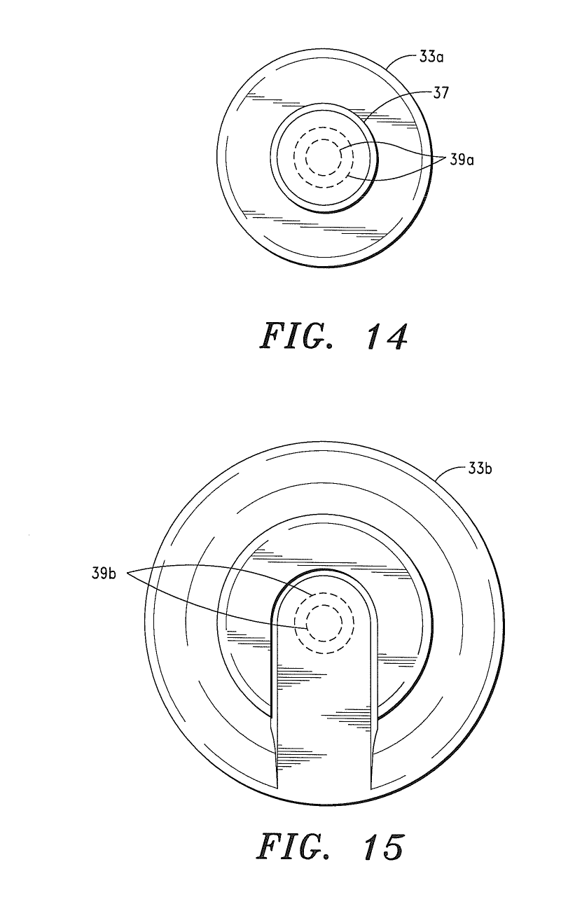

The top member 33a further includes an engagement end 37, which, as discussed below, is configured to seat in the bottom member 33b. Referring now to FIG. 14, in a preferred embodiment of the invention, the engagement end 37 of the top member 33a includes a plurality of conductive pads 39a.

The top member 33a further includes at least one, more preferably, a plurality of conductive circuit connection posts 39c that are in communication with the conductive pads 39a. In a preferred embodiment, the connection posts 39c, which are preferably disposed in the end opposing the engagement end 37, are configured to connect electronic circuits of a device associated therewith, e.g. module 40, to the top member 33a.

Referring back to FIG. 13, the bottom member 33b includes a spring clip 36 that is designed and configured to seat in spring seat 37, and removably engage the recessed region 37a of the engagement end 37 of the top member 33a when positioned in the bottom member 33b (see FIG. 12).

Referring to FIG. 15, the bottom member 33b also includes a plurality of conductive pads 39b that are disposed in the base of the bottom member 33b. The bottom member pads 39b are configured and positioned in the bottom member 33b, whereby the top member pads 39a and bottom member pads 39b are aligned when the top and bottom members 33a, 33b are connected, and whereby signals transmitted through the top member 33a are communicated to the bottom member 33b.

The bottom member 33b similarly includes at least one, more preferably, a plurality of conductive circuit connection posts 39d that are in communication with the conductive pads 39b. The connection posts 39d are configured to connect electronic circuits of a device associated therewith, e.g. garment band 105, to the bottom member 33b.

The bottom member 33b further includes a top member disengagement slot 38, which, as discussed below, is sized and configured to facilitate disengagement of the top member 33a from the bottom member 33b.

Referring now to FIG. 16, to engage the top and bottom members 33a, 33b, the top member 33a is positioned proximate to the bottom member 33b and moved in the direction denoted by arrows “E”. As the top and bottom members 33a, 33b move to a first spaced distance from each other, the force of the opposite polarity magnets 35a, 35b facilitates a secured seating of the engagement end 37 of the top member 33a in the bottom member 33b. The top member 33a is secured to the bottom member 33b via the spring clip 36, i.e. linear movement in a direction opposite arrows E is restricted.

Referring now to FIG. 17, to disengage the top and bottom members 33a, 33b the top member 33a is moved in the direction denoted by arrows “D”, whereby the engagement end 37 of the top member 33a transitions through the top member disengagement slot 38 in the bottom member 33b.

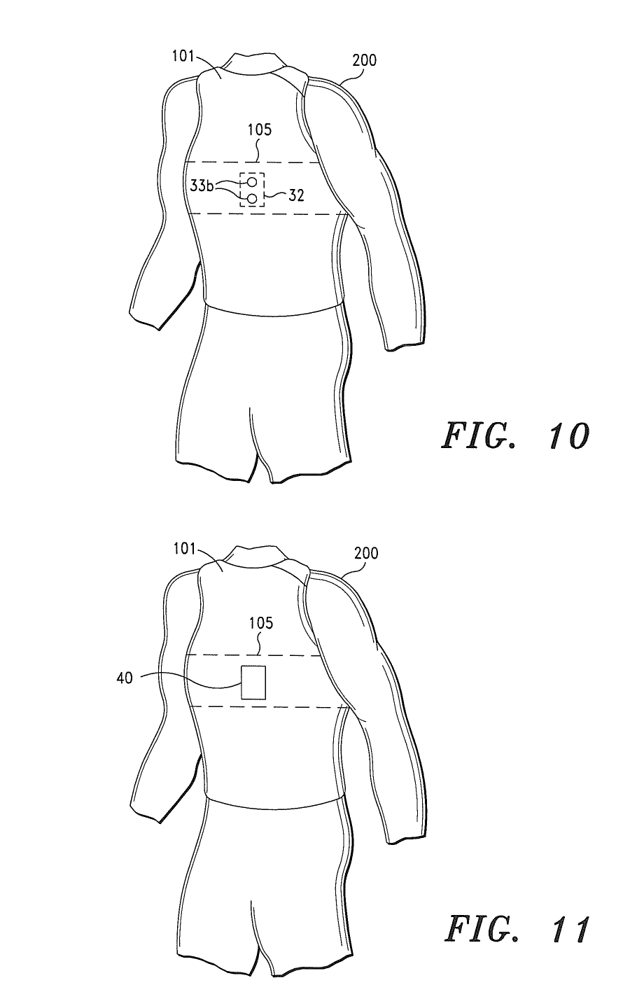

Referring now to FIGS. 9 and 10, in one embodiment of the invention, the first magnetic connector subsystem 32, which is associated with the system connection system 30, includes at least one connector member 33a, 33b, and the second magnetic connector 34, which is associated with the electronics module 34, includes at least one opposing member 33a or 33b.

In some embodiments of the invention, the first magnetic connector subsystem 32 includes at least one top member 33a and the second magnetic connector subsystem 34 includes at least one bottom member 33b.

In some embodiments, the first magnetic connector subsystem 32 includes at least one bottom member 33b and the second magnetic connector includes at least one top member 33a.

In some embodiments, the first magnetic connector subsystem 32 includes one top member 33a and one aligned bottom member 33b, and the second connector subsystem 34 includes mating bottom and top members 33b, 33a.

In the illustrated embodiment, the first magnetic connector subsystem 32 includes two spaced bottom members 33b and the second magnetic connector subsystem includes two similarly spaced top members 33a. In the noted embodiment, the top member disengagement slots 38 in the bottom members 33b are substantially aligned on a vertical or horizontal axis.

In the noted embodiment, the bottom member pads 39b are in communication with the band 105, and, hence, signal transmission conductors 10, and the top member pads 39a are in communication with the electronics module 40 electronics.

In a preferred embodiment, the magnets 35a that are disposed in the bottom members 33b have an opposite polarity, whereby only magnetic engagement of pared top and bottom members 33a, 33b can be achieved, and whereby proper connection of the connector pads 39a, 39b is ensured.

As will readily be appreciated by one having ordinary skill in the art, the present invention provides numerous advantages compared to prior art methods and systems for monitoring and/or detecting physiological characteristics. Among the advantages are the following:
The provision of an improved garment based physiological monitoring system and method that accurately (i) monitors and detects changes in (or displacements of) the anteroposterior diameters of the rib cage, and axial displacements of the chest wall, and (ii) determines anatomical and physiological information associated with the monitored subject as a function of the signals reflecting the noted anatomical displacements.
The provision of an improved garment based physiological monitoring system and method that accurately measures one or more additional physiological characteristics associated with a user or wearer, e.g. body temperature.
The provision of an improved garment based physiological monitoring system and method that does not require the user to secure electrodes to his/her body or to use any conductive gels.
The provision of an improved garment based physiological monitoring system and method that does not include any exposed electrical circuitry.
The provision of an improved garment based physiological monitoring system and method that does not include any wires which must be connected or routed by the wearer.
The provision of an improved garment based physiological monitoring system and method that includes reliable and effective means to connect external modules, e.g. processing units.
The provision of an improved garment based physiological monitoring system and method that does not interfere with the activities of or duties carried out by the user.
The provision of an improved garment based physiological monitoring system and method that requires minimal or no preparation prior to or after donning the garment.
The provision of an improved garment based physiological monitoring system and method that is easy to put on and is aesthetically pleasing.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A physiological monitoring system, comprising:
a garment that is configured to cover at least the chest region and the upper back of a wearer;
a stretchable circumferential band that is attachable to said garment, said stretchable band including a respiration detection system and integral signal transmission conductors in a flexible configuration thereon,
said respiration detection system including two magnetic coils that are secured and positioned by said garment, said magnetic coils being configured to monitor and detect axial chest wall displacements of said wearer;
an electronics module that is releasably attachable to said garment, said module including a processing system and a data transmission system, said processing system including programs, instructions and associated algorithms to control said respiration detection system, retrieve and process signals transmitted by said respiration detection system, determine physiological information associated with said wearer as a function of said respiration detection system signals, said data transmission system including a transmitter that is configured to wirelessly transmit said respiration detection system processed signals; and
a self-aligning magnetic connection system that is configured to removeably secure and couple said electronics module to said band, and provide a signal communication path between said electronics module and said band and, thereby, said signal transmission conductors.

2. The physiological monitoring system of claim 1, wherein said system includes a remote display unit.

3. The physiological monitoring system of claim 2, wherein said remote display unit includes a receiver that is configured to receive said respiration detection system processed signals from said electronics module.

4. The physiological monitoring system of claim 1, wherein said magnetic connection system includes a first and second magnetic connector subsystems, said first magnetic connector subsystem being attached to said band and in communication with said integral signal transmission conductors, said second magnetic connector subsystem being attached to said electronics module and in communication with said module circuitry.

5. The physiological monitoring system of claim 4, wherein said first magnetic connector subsystem that is accessible from outside said garment.

6. The physiological monitoring system of claim 1, wherein said system includes at least one additional physiological sensor that is in communication with said signal transmission conductors.

7. The physiological monitoring system of claim 6, wherein said at least one physiological sensor comprises a body temperature sensor.

8. The physiological monitoring system of claim 1, wherein said garment is constructed of Lycra®.
